(19) United States
(12) Patent Application Publication
(10) Pub. No.: US 2007/0225763 A1
(43) Pub. Date: Sep. 27, 2007

(54) Marked Suture

(75) Inventors: Andrew M. Zwolinski, Cincinnati, OH (US); Mark S. Ortiz, Milford, OH (US); Michael Stokes, Cincinnati, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Ethicon Endo-Surgery, Inc., Cincinnati, OH (US)

(21) Appl. No.: 11/277,283

(22) Filed: Mar. 23, 2006

(51) Int. Cl. A61L 17/00

(52) U.S. Cl. 606/228

(57) ABSTRACT

Disclosed herein is a suture material formed of a flexible strand of suture having at least one marking thereon. The marking is one that is indicative of at least one use characteristic of the suture.

### FIELD OF THE INVENTION

The present invention relates broadly to an improved suture material, in particular, a suture material that includes at least one marking.

### BACKGROUND OF THE INVENTION

It is well known that suture material is commonly used to repair openings in skin, internal organs, blood vessels, and a variety of other tissues of the human body. Conventionally, suturing of human tissue occurs during open surgery or minimally invasive surgical procedures.

In certain minimally invasive surgical procedures, e.g., endoscopic and laparoscopic surgeries, a surgeon performs diagnostic and therapeutic procedures at the surgical site through a natural body aperture or through one or more small incisions, using instruments specially designed for this purpose. Problems encountered by a surgeon in such minimally invasive surgical procedures include reduced visibility and field of vision, as well as potential orientation difficulties when performing the required manipulations at the surgical site, such as suturing tissue.

Suturing procedures can be particularly challenging in minimally invasive surgical procedures. For example, it can be difficult for a surgeon to determine various properties of the suture material being used, such as the amount of suture used, direction of the suture, and the forces to which the suture is subjected. As a result, use of such suture material adds to the visibility and orientation problems encountered by a surgeon when suturing in a minimally invasive surgical procedure.

Accordingly, a need exists for an improved suture material that provides enhanced ease of use, particularly in minimally invasive surgical procedures.

### SUMMARY OF THE INVENTION

The present invention generally provides an improved suture material for enhanced ease of use, particularly in minimally invasive surgical procedures. The suture material can include a flexible strand of suture that can have formed thereon at least one marking. The marking can provide information on the various properties of the suture, such as indicating a at least one use characteristic of the suture. Examples of suture use characteristics include suture orientation, direction of suture motion, amount of suture, length of suture, depth of suture penetration, as well the tension being applied on a suture.

In one embodiment, a marking on the suture can include one or more arrow-like indicia that can be formed in a spaced relationship along a length of the suture and, optionally, about a circumference of the suture.

In another embodiment, a marking on the suture can include a plurality of graduations. The graduations can be formed around at least a portion of a circumference of the suture and be spaced from each other along a length of the suture. The graduations can also be spaced at regular, predetermined intervals along a length of the suture and, by way of example, can provide a scale to aid in estimating a dimension of length of the suture. Alternatively, the graduations can be spaced at increasing increments along a length of the suture. Such graduations can provide a qualitative or quantitative indication of the progression, or depth of penetration, of the suture through the material being sutured. The increase or decrease in the spacing of the graduations can also be indicative of direction.

In a further exemplary embodiment, a marking on the suture can include parallel lines extending along all or a portion of the length of the suture. The parallel lines can provide information as to the tension being exerted on the suture. For example, when a length of suture is stretched, the parallel lines can converge or deviate from the parallel, thereby indicating the stress or strain on the suture.

A method for suturing with suture material according to the aspects of the present invention is also provided. The method includes the steps of providing a length of suture having formed thereon at least one marking indicative of a use characteristic of the suture, and passing the suture through tissue that is to be sutured while observing a use characteristic of the suture.

### BRIEF DESCRIPTION OF THE DRAWINGS

The invention will be more fully understood from the following detailed description taken in conjunction with the accompanying drawings, in which:

FIG. 1 presents a perspective view of a prior art strand of suture material;

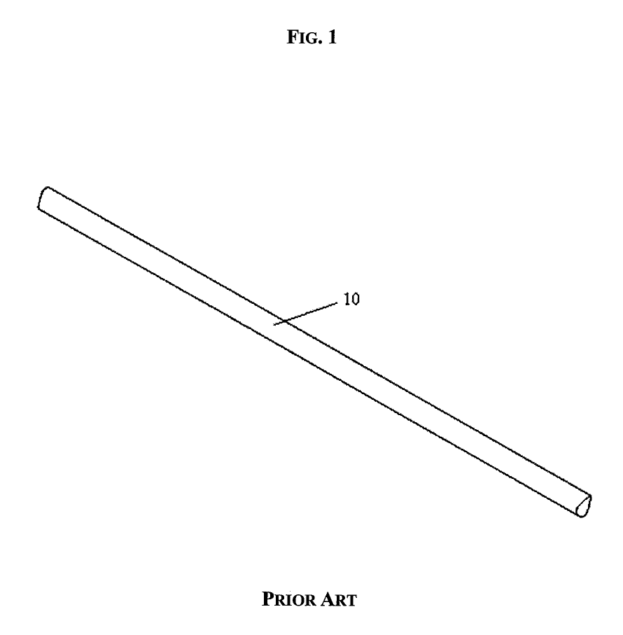

FIG. 2 presents a perspective view of suture materials constructed according to aspects of the present invention;

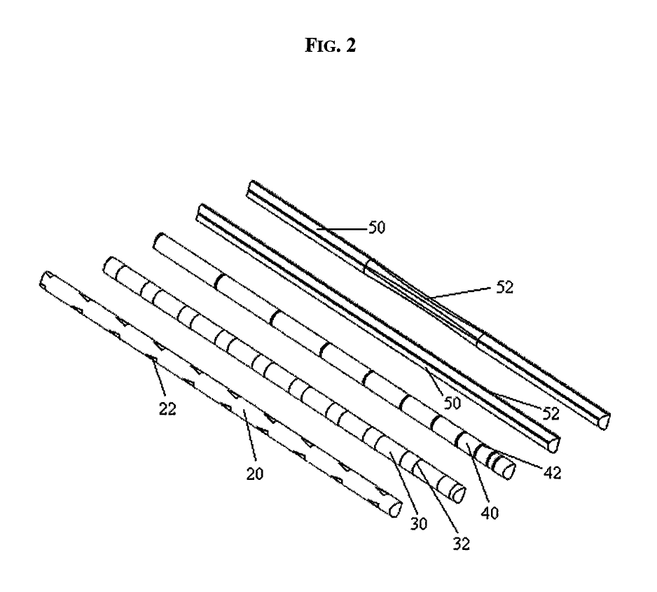

FIG. 3 presents a perspective view of a strand of suture having formed thereon markings that are comprised of arrow-like indicia;

FIG. 4 presents a perspective view of a strand of suture having formed thereon markings that are comprised of graduations spaced at regular, pre-determined intervals along the length of the suture;

FIG. 5 presents a perspective view of a strand of suture having formed thereon markings that are comprised of graduations spaced at increasing increments along the length of the suture;

FIG. 6 presents a perspective view of a strand of suture having formed thereon markings that are comprised of parallel longitudinal lines extending along the suture;

FIG. 7 presents a perspective view of the suture shown in FIG. 6, when tension is applied to the suture.

### DETAILED DESCRIPTION OF THE INVENTION

Certain exemplary embodiments will now be described to provide an overall understanding of the principles, structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides an improved suture material that includes a flexible strand of suture that can have formed thereon at least one marking. The marking is adapted to provide various informational properties of the suture to a user of the improved suture material. For example, a marking can indicate information relating to at least one use characteristic of the suture. Examples of use characteristics include: how a suture is orientated, the direction of the suture, a length of the suture, an amount of the suture, the direction of movement by the suture, the progression of the suture, the depth of penetration of the suture, and whether tension is being exerted on the suture. In use, the improved suture material is passed through tissue that is being sutured, and a user is able to discern at least one use characteristic of the suture based on the indications provided by at least one marking that is formed on the suture. The improved suture material is advantageous in that it provides an enhanced ease of use of a suture, and facilitates in alleviating some of the visibility and orientation problems encountered when suturing tissue, particularly in a minimally invasive surgical procedure.

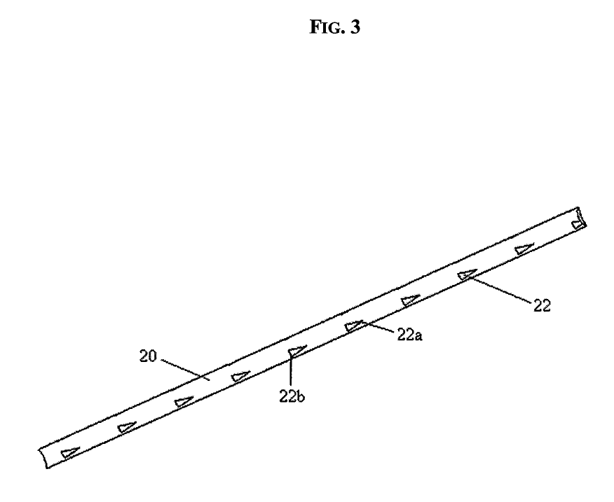

Referring to FIG. 3, one example of an improved suture material of the present invention is shown having a flexible strand of suture 20 which has formed thereon at least one marking. The marking on suture 20 can be a plurality of arrow-like indicia 22. As shown, each arrow-like indicia 22 has a point 22a and a base 22b. The points 22a of the arrow-like indicia 22 may, for example, be oriented so as to point in a desired direction of travel of suture 20. The arrow-like indicia 22 can be formed in a spaced relationship along a length of suture 20. In addition, indicia 22 can be formed in a single row or in a spaced relationship around a circumference of suture 20, such as in substantially parallel rows about the circumference of the suture. One skilled in the art will appreciate that the spacing intervals can vary. For example, the arrow-like indicia 22 can be spaced at intervals in the range of about 5 mm to 20 mm.

When suture 20 is in use, arrow-like indicia 22 can provide an indication of the direction in which suture 20 is orientated. The arrow-like indicia 22 can also be indicative of the direction of motion of suture 20. In a further example, the arrow-like indicia 22 can indicate the direction suture 20 needs to be pulled for loosening or tightening the suture. In an alternative example, the arrow-like indicia 22 can aid in orientating the user employing the suture.

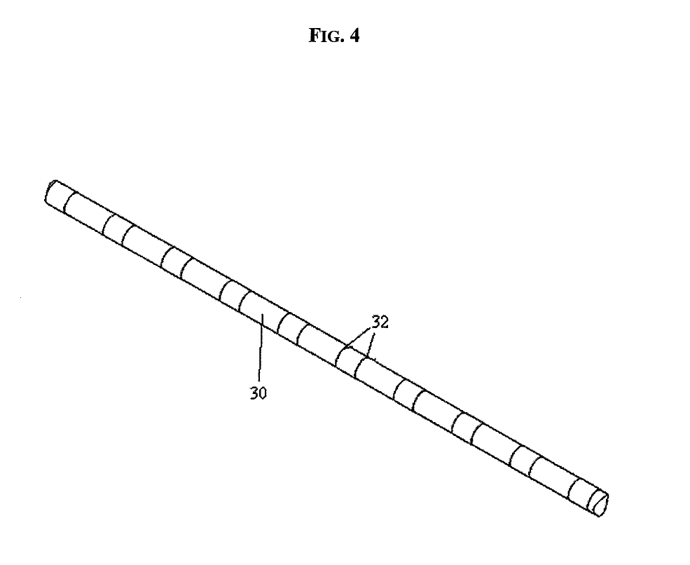

FIG. 4 illustrates another embodiment of the suture material of the present invention having formed thereon a plurality of graduations 32. The graduations 32 can be formed around at least a portion of the circumference of a suture 30. Graduations 32 can also be formed around the entire portion of the circumference of suture 30, in a continuous manner or in a broken manner. The graduations 32 can be spaced from each other along a length of suture 30 at regular, predetermined intervals. One skilled in the art will appreciate that the spacing in the intervals can vary. For example, graduations 32 can be spaced from each other at intervals in the range about 5 mm to 20 mm. In addition, one skilled in the art will appreciate that the thickness of the graduations 32 can vary.

Graduations 32 can be indicative of a variety of use characteristics of suture 30. For example, they can by provide a scale of suture 30. That is, based on a known spacing of the graduations 32, a user of suture 30 can count the number of graduations 32 to arrive at an estimation of a length dimension. Further, the graduations 32 can provide a scale of suture 30 that enables an estimation of suture size or suture amount.

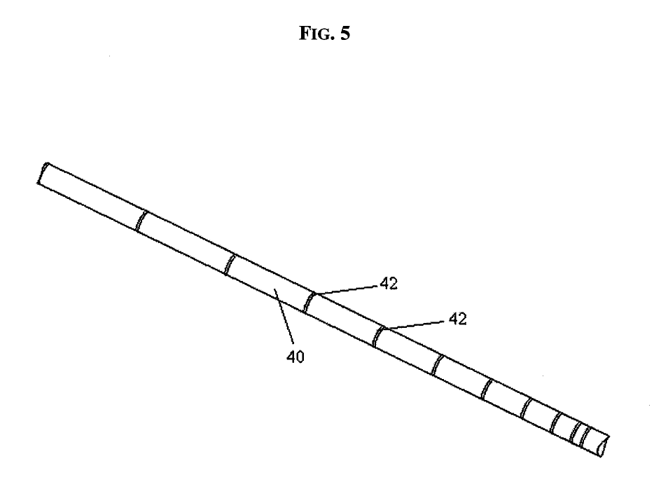

FIG. 5 shows a further embodiment of the improved suture material having formed thereon a plurality of graduations 42. The graduations 42 can be formed around at least a portion of the circumference of a suture 40. Graduations 42 can also be formed around the entire portion of the circumference of suture 40, in a continuous manner or in a broken manner. The graduations 42 can be spaced at increasing increments along the length of suture 40. For example, the graduations can be rather closely spaced (e.g., about 1 mm) at a proximal end of the suture and have increasingly greater spacing intervals moving distally along the suture. One skilled in the art will appreciate that the thickness of the graduations 42 can vary.

Graduations 42 can be indicative of a use characteristic of suture 40 by providing a user with qualitative information or a quantitative value. For example, the increasing increments of the graduations 42 can provide qualitative information or a quantitative value that is indicative of the length of progression of suture 40 through tissue being sutured. In a further example, graduations 42 can provide a qualitative or quantitative indication of the depth of penetration by suture 40 through tissue being sutured. In another example, graduations 42 can provide an indication of direction based on whether the incremental spacing of graduations 42 is increasing or decreasing.

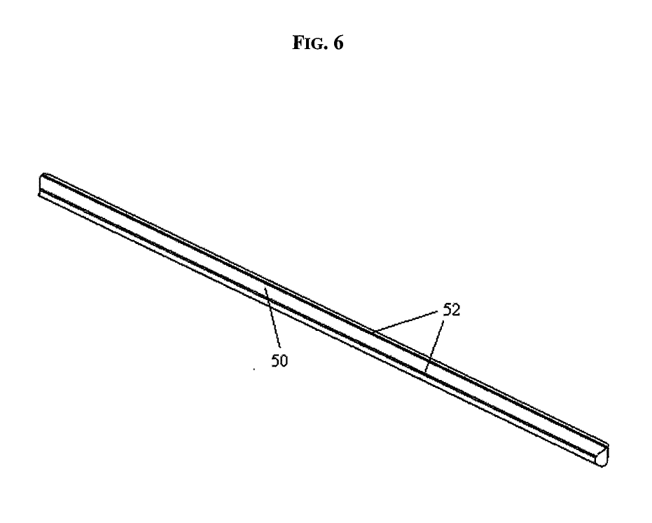

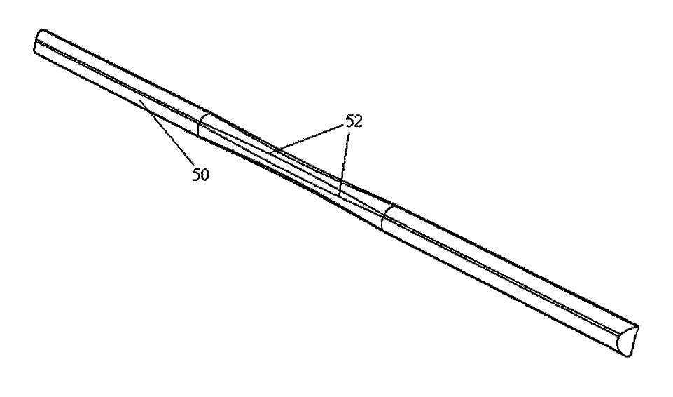

Another exemplary embodiment of the suture material made according to the present invention is shown in FIGS. 6 and 7. The suture material depicted in FIGS. 6 and 7, can be a flexible strand of suture 50 having formed thereon at least two parallel longitudinal lines 52, which can extend along at least a portion of the length of suture 50. Lines 52 can be continuous or they can be broken lines.

Parallel longitudinal lines 52 can provide information on a use characteristic of suture 50, such as whether tension is being exerted upon suture 50. As shown in FIG. 6, when suture 50 is not under tension, lines 52 run parallel to one another along the length of the suture. When suture 50 is subjected to tension, however, the lines 52 to converge or deviate from parallel, as depicted in FIG.7. For example, when a user pulls upon suture 50 such as during suturing of tissue, or when sutured tissue pulls upon suture 50, the suture 50 is stretched such that lines 52 can converge or deviate from the parallel, and thus can provide an indication that stress or strain is being applied to suture 50.

One skilled in the art will appreciate that the suture material of the present invention can be otherwise constructed to indicate other use characteristics of the suture material and/or to enhance visibility of the suture. For example, at least one marking formed on a flexible strand of suture as described herein can be radiopaque. Alternatively, the markings on a suture described herein can be of a contrasting color. For example, at least one marking can be of a color other than the color of the suture. Further, the markings formed on the sutures described herein can be of a texture different from a texture of the suture. For example, the markings can be indented in the strand of suture, or raised on the suture strand. In addition, a person skilled in the art will appreciate that the improved suture material of the present invention can include any combination of markings or features described herein, as well as other markings or features known in the art.

The suture material of the present invention can be used in a variety of surgical procedures, for example, for repairing or re-attaching or torn incised tissue. In an exemplary embodiment, the suture material can be used in a suturing procedure conducted during a minimally invasive surgical procedure, such as an endoscopic or laparoscopic procedure. In another exemplary embodiment, the suture material is used in the context of stitching epidermal disruptions. It will be understood, however, that the method described herein is equally applicable to connecting detached tissue in other contexts as well, such as during open and invasive surgical procedures.

A method for suturing with the suture material described herein includes providing a length of suture that can have formed thereon at least one marking which can be indicative of a use characteristic of the suture. In one exemplary technique for using the improved suture material, a length of a flexible strand of suture 20 is provided having formed thereon arrow-like indicia 22, and suture 20 is passed through tissue that is being sutured. The arrow-like indicia 22 provide a user with an indication of a use characteristic, for example, the direction of orientation of suture 20.

The improved suture material of the present invention can be formed from a variety of known materials that are suitable for use in forming suture material. For example, the suture can be made from a variety of polymers that can be resorbable or non-resorbable.

The embodiments of the present invention can be formed onto the suture material in a variety of processes. By way of example, the various suture markings embodying the present invention can be applied to the sutures described herein by an off-set printing process. The markings can also be applied to the sutures of the present invention by a pad printing process, which can involve obtaining an ink from a master, and subsequently transferring the impression to the suture material.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present invention. As well, one skilled in the art will appreciate further features and advantages of the invention based on the above-described embodiments. Accordingly, the invention is not to be limited by what has been particularly shown and described, except as indicated by the appended claims.

What is claimed is:

1. A suture material comprising:
a flexible strand of suture having formed thereon at least one marking indicative of a use characteristic of the suture;
wherein the use characteristic is at least one of suture orientation, suture direction, suture length, suture amount, direction of suture motion, depth of suture penetration, and suture tension.

2. The suture material of claim 1, wherein at least one marking comprises a plurality of arrow-like indicia.

3. The suture material of claim 2, wherein the arrow-like indicia are formed in a spaced relationship along a length of the suture.

4. The suture material of claim 3, wherein the arrow-like indicia are formed in parallel rows about the circumference and along the length of the suture.

5. The suture material of claim 2, wherein the arrow-like indicia are indicative of the use characteristic of direction of suture orientation.

6. The suture material of claim 2, wherein the arrow-like indicia are indicative of the use characteristic of direction of suture motion.

7. The suture material of claim 1, wherein at least one marking comprises a plurality of graduations.

8. The suture material of claim 7, wherein the graduations are formed around at least a portion of a circumference of the suture and spaced from each other along a length of the suture.

9. The suture material of claim 7, wherein the graduations are spaced at regular, predetermined intervals along a length of the suture.

10. The suture material of claim 9, wherein the graduations spaced at regular intervals are indicative of the use characteristic of suture scale for estimation of a length dimension.

11. The suture material of claim 7, wherein the graduations are spaced at increasing increments along a length of the suture.

12. The suture material of claim 11, wherein the graduations spaced at increasing increments provide a qualitative or quantitative indication of the use characteristic of length of suture progression.

13. The suture material of claim 11, wherein the graduations spaced at increasing increments provide a qualitative or quantitative indication of the use characteristic of depth of suture penetration.

14. The suture material of claim 11, wherein the graduations spaced at increasing increments are indicative of the use characteristic of suture orientation.

15. The suture material of claim 1, wherein at least one marking comprises at least two parallel longitudinal lines extending along a length of the suture.

16. The suture material of claim 15, wherein a characteristic of the parallel lines is indicative of a stress or strain characteristic of the suture.

17. The suture material of claim 1, wherein at least one marking is radiopaque.

18. The suture material of claim 1, wherein at least one marking is of a different texture than a texture of the suture.

19. A method of suturing, comprising:
providing a length of suture having formed thereon at least one marking indicative of a use characteristic of the suture; and
passing the suture through tissue that is to be sutured.

20. The method of claim 19, wherein the suturing is performed endoscopically.

21. The method of claim 19, wherein the suturing is performed laparoscopically.

22. The method of claim 19, wherein the suturing is performed open, or stitching epidermal disruptions.
